SEQUENCE LISTING (4 sequences)

SEQ ID NO: 1 - protein, 8 aa, Artificial Sequence
  Synthetic construct
FCFWKTCT

SEQ ID NO: 2 - protein, 8 aa, Artificial Sequence
  Synthetic construct
XCYWKVCT

SEQ ID NO: 3 - protein, 8 aa, Artificial Sequence
  Synthetic construct
TEEEQQYL

SEQ ID NO: 4 - protein, 9 aa, Artificial Sequence
  Synthetic construct
EKTXVNXGN

(19) United States
(12) Patent Application Publication
(10) Pub. No.: US 2013/0034597 A1
(43) Pub. Date: Feb. 7, 2013

(54) ORALLY BIOAVAILABLE PEPTIDE DRUG COMPOSITIONS AND METHODS THEREOF

(71) Applicant: Edward T. Maggio, San Diego, CA (US)

(72) Inventor: Edward T. Maggio, San Diego, CA (US)

(73) Assignee: AEGIS THERAPEUTICS LLC, San Diego, CA (US)

(21) Appl. No.: 13/366,108

(22) Filed: Feb. 3, 2012

Related U.S. Application Data
(60) Provisional application No. 61/450,547, filed on Mar. 8, 2011.
(60) Provisional application No. 61/439,711, filed on Feb. 4, 2011.

(51) Int. Cl. A61K 38/12; A61K 38/03; A61K 38/16; A61P 31/00; A61P 9/08; A61K 9/127; A61K 38/43; A61K 38/02; A61K 38/14

(52) U.S. Cl. 424/450; 514/21.1; 514/2.3; 424/94.1

(57) ABSTRACT

The present invention provides orally bioavailable peptide drug compositions including a cyclic peptide and an orally compatible absorption enhancer, as well as methods for providing increased oral bioavailability of peptide drugs.

### CROSS REFERENCE TO RELATED APPLICATION(S)

This application claims the benefit of priority under 35 U.S.C. §119(e) of U.S. Ser. No. 61/450,547, filed Mar. 8, 2011; and the benefit of priority under 35 U.S.C. §119(e) of U.S. Application Ser. No. 61/439,711, filed Feb. 4, 2011. The entire contents of each of the prior applications is incorporated herein by reference in their entirety.

### BACKGROUND OF THE INVENTION

1. Field of the Invention

The invention relates generally to oral compositions and more specifically to orally bioavailable peptide drug compositions including a cyclic peptide and an orally compatible absorption enhancer, as well as methods for providing increased oral bioavailability of peptide drugs.

2. Background Information

In spite of the many attractive aspects of peptides and proteins as potential therapeutic agents, their susceptibility to denaturation and hydrolysis in the gastrointestinal tract makes them unsuitable for oral administration, and this remains their major shortcoming as drugs. Therefore, while the range of clinical indications for therapeutic proteins and peptides is quite broad, the actual number of such therapeutics in general use today is quite small compared with the number of chemically synthesized and orally active pharmaceuticals currently on the market. Most peptide therapeutics are administered by injection save for a few exceptions. Injection is an inconvenient and expensive mode of administration. For situations where the medical consequences may not be immediate or life-threatening, and in cases where the administration must be frequent and chronic, patient noncompliance naturally becomes a serious issue. Extended half-life derivatives (i.e. via pegylation) and depot formulations of peptide and protein therapeutics, both still requiring injection but at a reduced frequency, are partial but imperfect solutions and bring with them their own set of pharmacological problems and limitations.

For some peptide therapeutics, intranasal delivery has proven to be an acceptable route of administration. However, bioavailability, even for small peptides such as calcitonin (less than 4 kDa) which must be administered chronically on a daily basis for treatment of osteoporosis, is only about 3% on average. Nevertheless, the advantages of intranasal administration in terms of greater patient comfort, convenience, and elimination of needlestick injuries and syringe disposal concerns associated with daily injections, far outweighs the higher manufacturing costs resulting from poor bioavailability of current intranasal formulations. This is clearly evidenced by the commercial success of intranasal calcitonin. From a technical perspective, however, success in intranasal delivery of peptides continues to be less than satisfactory in most cases, and the previously cited average bioavailability of 3% for calcitonin, with broad patient to patient variability ranging from 0.3% to 30.6%, has actually been among the best performances for intranasal delivery of peptides.

A large number of molecules have been screened for their ability to enhance transmucosal absorption. Some molecules have been found to increase transmucosal absorption of peptides across the nasal mucosa and across the rectal mucosa. However, the oral absorption of peptides has remained essentially an elusive goal. Further, only some classes of absorption enhancers are orally compatible. Many cause damage to the gastrointestinal tract. For example, administration of taurocholic acid causes decapitation of the intestinal pilli found in the gastrointestinal tract.

### SUMMARY OF THE INVENTION

The present invention provides oral compositions which exhibit increased oral bioavailable of peptide drugs. As such, in one aspect, the present invention provides an oral composition including a peptide. The oral composition includes a cyclic peptide; and at least one alkylsaccharide absorption enhancer.

In another aspect, the present invention provides a method of increasing the oral bioavailability of a linear peptide. The method includes cyclizing a linear peptide to form a cyclic peptide; and orally administering the cyclized peptide in the presence of at least one alkylsaccharide absorption enhancer to a subject. In some embodiments, the cyclic peptide and the at least one alkylsaccharide are admixed to form a composition prior to oral administration.

### BRIEF DESCRIPTION OF THE DRAWINGS

FIG. 1 is a graphical representation plotting an octreotide acetate uptake profile following subcutaneous delivery in sodium acetate buffer. The graph depicts serum concentrations of octreotide acetate at 5, 10, 15, 30, 60, 120 and 180 minutes after subcutaneous delivery of 30 mcg in sodium acetate buffer to male Swiss Webster mice (n=3 mice per time point). Each value represents mean+SEM octreotide acetate concentration. Error bars are contained within each point and ranged between 0.01 and 0.10 ng/ml.

FIG. 2 is a graphical representation plotting an octreotide acetate uptake profile following oral delivery (by gavage) in 0.5% n-dodecyl-beta-D-maltoside (DDM). The graph depicts serum concentrations of octreotide acetate at 5, 10, 15, 30, 60, 120 and 180 minutes after oral delivery (by gavage) of 30 mcg in 0.5% DDM to male Swiss Webster mice (n=3 mice per time point). Each value represents mean+SEM octreotide acetate concentration. Error bars are contained within each point and ranged between 0.01 and 0.1 ng/ml.

FIG. 3 is a graphical representation plotting an octreotide acetate uptake profile following oral delivery (by gavage) in 1.5% DDM. The graph depicts serum concentrations of octreotide acetate at 5, 10, 15, 30, 60, 120, and 180 minutes after oral delivery (by gavage) of 30 mcg in 1.5% DDM to male Swiss Webster mice (n=3 mice per time point). Each value represents mean+SEM octreotide acetate concentration. Error bars are contained within each point and ranged between 0.01 and 0.10 ng/ml.

FIG. 4 is a graphical representation plotting an octreotide acetate uptake profile following oral delivery (by gavage) in 3.0% DDM. The graph depicts serum concentrations of octreotide acetate at 5, 10, 15, 30, 60, 120 and 180 minutes after oral delivery (by gavage) of 30 mcg in 3.0% DDM to male Swiss Webster mice (n=3 mice per time point). Each value represents mean+SEM octreotide acetate concentration. Error bars are contained within each point and ranged between 0.01 and 0.10 ng/ml.

### DETAILED DESCRIPTION OF THE INVENTION

While linear peptides are poorly absorbed orally, whether or not an absorption enhancer is present, in the present invention, it has been discovered that a particular structural class of peptides including small to intermediate length cyclic peptides, when combined with orally compatible absorption enhancers give rise to compositions with dramatically increased oral bioavailability. Further, it has been discovered that incorporation of so-called non-natural amino acids into a cyclic peptide may further enhance the oral bioavailability of compositions containing such cyclic peptides and an orally compatible absorption enhancer while preserving biological activity.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present invention provides therapeutic compositions which exhibit increased oral bioavailable of peptide drugs. The oral composition of the present invention includes a cyclic peptide and at least one alkylsaccharide absorption enhancer.

Further provided is a method of increasing the oral bioavailability of a linear peptide. The method includes cyclizing a linear peptide to form a cyclic peptide; and orally administering the cyclized peptide in the presence of at least one alkylsaccharide absorption enhancer to a subject.

As used herein, a cyclized peptide refers to a peptide that is generally cyclic in structure as a result of a linkage between two amino acids. Further, the terms “cyclic” and “cyclized” are used synonymously and refer to a peptide that has been synthetically cyclized or naturally occurs as a cyclic protein.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-natural amino acid. Additionally, such “polypeptides,” “peptides” and “proteins” include amino acid chains of any length, including full length proteins, wherein the amino acid residues are linked by covalent peptide bonds.

Cyclic peptides as disclosed in several embodiments of this invention may be readily synthesized by any known conventional procedure for the formation of a peptide linkage between amino acids. Such conventional procedures include, for example, any solution phase procedure permitting a condensation between the free alpha amino group of an amino acid residue having its carboxyl group or other reactive groups protected and the free primary carboxyl group of another amino acid residue having its amino group or other reactive groups protected.

The process for synthesizing the cyclic peptides may be carried out by a procedure whereby each amino acid in the desired sequence is added one at a time in succession to another amino acid residue or by a procedure whereby peptide fragments with the desired amino acid sequence are first synthesized conventionally and then condensed to provide the desired peptide. The resulting peptide is then cyclized to yield a cyclic peptide of the invention. A cyclic peptide can be obtained by inducing the formation of a covalent bond between an amino group at the N-terminus of the peptide, if provided, and a carboxyl group at the C-terminus, if provided. A cyclic peptide can also be obtained by forming a covalent bond between a terminal reactive group and a reactive amino acid side chain moiety, or between two reactive amino acid side chain moieties. One skilled in the art would know that the means by which a given peptide is made cyclic is determined by the reactive groups present in the peptide and the desired characteristic of the peptide.

The cyclic peptides for use with the present invention are of a particular structural class which includes small to intermediate length cyclic peptides. Such peptides when combined with an alkylsaccharide absorption enhancer gives rise to compositions with dramatically increased oral bioavailability. Cyclic peptides of the present invention may include from 2 to 50 amino acids, for example 3, 4, 5, 6, 7, 8, 9, 10, 11, 12, 13, 14 or 15 up to 50 amino acids, including 3, 4, 5, 6, or 7 up to 10, 15, 20, 25, 30, 35, 40, 45 or 50 amino acids. In some embodiments the peptide includes 2 to 20 amino acids, for example 5 to 15 amino acids, 5 to 13 amino acids, 7 to 13 amino acids, or 8 to 12 amino acids. In some embodiments, the peptide includes less than 50, 45, 40, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6 or 5 amino acids.

The cyclic peptide may be cyclized from any desired linear peptide, or alternatively occur naturally in a cyclic form. In some embodiments the cyclic peptide is selected from SEQ ID NOs: 1-4. In some embodiment the cyclic peptide is an antibiotic. As used herein a “cyclic peptide antibiotic” refers to a cyclic peptide which demonstrates antimicrobial activity. Examples of cyclic peptide antibiotics useful in the present invention include, but are not limited to daptomycin, vancomycin, bacitracin, gramicidin, grandamycin, viomycin, capreomycin, microcin J25, bacteriocin AS-48, rhesus theta defensin-1 (RTD-1), streptogramins, and polymyxins, such as polymyxin B, E and M.

It has been discovered that inclusion of non-natural amino acids into the cyclic peptide enhance oral bioavailability when administered with an alkysaccharide. As such, cyclic peptides of the present invention may include at least one non-natural amino acid. One skilled in the art would understand that a non-natural amino acid may be incorporated by a variety of methods known in the art, such as by addition, or alternatively by substitution or modification of an exiting amino acid. As such, a cyclic peptide of the invention may include at least 10%, 20%, 30%, 40%, 50%, 60%, 70%, 80% or 90% of natural or L-amino acids, with the remainder being non-natural. For example, the cyclic peptide may include at least 90%, 91%, 92%, 93%, 94%, 95%, 96%, 97%, 98% or 99% natural amino acids.

A “non-natural amino acid” refers to an amino acid that is not one of the 20 common amino acids, namely alanine, cysteine, aspartic acid, glutamic acid, phenylalanine, lysine, histidine, isoleucine, lysine, leucine, methionine, asparagine, proline, glutamine, arginine, serine, threonine, valine, tryptophan, and tyrosine, or pyrolysine or selenocysteine. Other terms that may be used synonymously with the term “non-natural amino acid” is “non-naturally encoded amino acid,” “unnatural amino acid,” “non-naturally-occurring amino acid,” and variously hyphenated and non-hyphenated versions thereof. The term “non-natural amino acid” includes, but is not limited to, amino acids which occur naturally by modification of a naturally encoded amino acid (including but not limited to, the 20 common amino acids or pyrrolysine and selenocysteine) but are not themselves incorporated into a growing polypeptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine. Additionally, the term “non-natural amino acid” includes, but is not limited to, amino acids which do not occur naturally and may be obtained synthetically or may be obtained by modification of non-natural amino acids.

A polypeptide including a non-natural amino acid may be produced biosynthetically or non-biosynthetically. By biosynthetically is meant any method utilizing a translation system (cellular or non-cellular), including use of at least one of the following components: a polynucleotide, a codon, a tRNA, and a ribosome. By non-biosynthetically is meant any method not utilizing a translation system: this approach can be further divided into methods utilizing solid state peptide synthetic methods, solid phase peptide synthetic methods, methods that utilize at least one enzyme, and methods that do not utilize at least one enzyme; in addition any of this sub-divisions may overlap and many methods may utilize a combination of these sub-divisions.

For purposes herein, non-natural amino acids can include amino acids containing the D-isomer configuration since most proteins are comprised primarily or entirely of amino acids in the L-isomer configuration, notwithstanding the fact that D-amino acids do occur naturally in certain situations, including, for example, bacterial, fungal, and plant metabolism and byproducts.

Peptides containing non-natural amino acids, such as D-amino acids and those including substituted side chains exhibit improved stability in the gastrointestinal tract as a result of reduced proteolysis. As such, the non-natural amino acid may be further modified. For instance, the sidechain of a non-natural amino acid component(s) of a polypeptide can provide a wide range of additional functionality to the polypeptide. By way of example only, and not as a limitation, the sidechain of the non-natural amino acid portion of a polypeptide may include any of the following: a label; a dye; a polymer; a water-soluble polymer; a derivative of polyethylene glycol; a photocrosslinker; a cytotoxic compound; a drug; an affinity label; a photoaffinity label; a reactive compound; a resin; a second protein or polypeptide or polypeptide analog; an antibody or antibody fragment; a metal chelator; a cofactor; a fatty acid; a carbohydrate; a polynucleotide; a DNA; a RNA; an antisense polynucleotide; a saccharide, a water-soluble dendrimer, a cyclodextrin, a biomaterial; a nanoparticle; a spin label; a fluorophore, a metal-containing moiety; a radioactive moiety; a novel functional group; a group that covalently or noncovalently interacts with other molecules; a photocaged moiety; an actinic radiation excitable moiety; a ligand; a photoisomerizable moiety; biotin; a biotin analogue; a moiety incorporating a heavy atom; a chemically cleavable group; a photocleavable group; an elongated side chain; a carbon-linked sugar; a redox-active agent; an amino thioacid; a toxic moiety; an isotopically labeled moiety; a biophysical probe; a phosphorescent group; a chemiluminescent group; an electron dense group; a magnetic group; an intercalating group; a chromophore; an energy transfer agent; a biologically active agent; a detectable label; a small molecule; an inhibitory ribonucleic acid, a radionucleotide; a neutron-capture agent; a derivative of biotin; quantum dot(s); a nanotransmitter; a radiotransmitter; an abzyme, an activated complex activator, a virus, an adjuvant, an aglycan, an allergan, an angiostatin, an antihormone, an antioxidant, an aptamer, a guide RNA, a saponin, a shuttle vector, a macromolecule, a mimotope, a receptor, a reverse micelle, and any combination thereof.

Examples of some non-natural amino acid substitutions include, but are not limited to, replacement of L-amino acids with D-amino acids. Other examples include replacement of naturally occurring aminoacyl chains with derivatized chains, for example, substituting hydroxyproline for proline. Other non-natural amino acids which may be substituted include norleucine, tert-leucine, hydroxyvaline, allothreonine, beta, beta-dialkylserine, cyclohexylalanine, allylglycine, napthylalanine, pyridylalanine, 4-hydroxymphenylglycine, phenylglycine, homoserine, 3,4,dihydroxyphenylalanine, 4-chlorophenylalanine.

As discussed herein, oral administration of a cyclic peptide as disclosed in combination with an alkylsaccharide penetration enhancer, increases the oral bioavailability of the peptide. As used herein, “alkylsaccharide” refers to any sugar joined by a linkage to any hydrophobic alkyl, as is known in the art. The alkylsaccharide is nonionic as well as nontoxic and considered Generally Recognized As Safe, for food applications, sometimes referred to as a GRAS substance. Alkylsaccharides are available from a number of commercial sources and may be natural or synthesized by known procedures, such as chemically or enzymatically. An absorption enhancer considered to be orally compatible is one which does not cause severe or irreversible damage to gastrointestinal tissues.

In various aspects, alkylsaccharides of the present invention may include, but not limited to: alkylglycosides, such as octyl-, nonyl-, decyl-, undecyl-, dodecyl-, tridecyl-, tetradecyl-, pentadecyl-, hexadecyl-, heptadecyl-, and octadecyl-α- or β-D-maltoside, -glucoside or -sucroside; alkyl thiomaltosides, such as heptyl, octyl, dodecyl-, tridecyl-, and tetradecyl-β-D-thiomaltoside; alkyl thioglucosides, such as heptyl- or octyl 1-thio α- or β-D-glucopyranoside; alkyl thiosucroses; alkyl maltotriosides; long chain aliphatic carbonic acid amides of sucrose β-amino-alkyl ethers; derivatives of palatinose and isomaltamine linked by amide linkage to an alkyl chain; derivatives of isomaltamine linked by urea to an alkyl chain; long chain aliphatic carbonic acid ureides of sucrose β-amino-alkyl ethers; and long chain aliphatic carbonic acid amides of sucrose β-amino-alkyl ethers.

As described above, the hydrophobic alkyl can thus be chosen of any desired size, depending on the hydrophobicity desired and the hydrophilicity of the saccharide moiety. For example, one preferred range of alkyl chains is from about 10 to about 24 carbon atoms. An even more preferred range is from about 10 to about 16 or about 14 carbon atoms. Similarly, some preferred glycosides include maltose, sucrose, and glucose linked by glycosidic linkage to an alkyl chain of 9, 10, 12, 13, 14, 16, 18, 20, 22, or 24 carbon atoms, for example, nonyl-, decyl-, dodecyl-, tridecyl, and tetradecyl sucroside, glucoside, maltoside, and the like. These compositions are nontoxic, since they are degraded to an alcohol or fatty acid and an oligosaccharide, and amphipathic. Additionally, the linkage between the hydrophobic alkyl group and the hydrophilic saccharide can include, among other possibilities, a glycosidic, thioglycosidic, amide, ureide, or ester linkage.

In sugar chemistry, an anomer is either of a pair of cyclic stereoisomers (designated α or β) of a sugar or glycoside, differing only in configuration at the hemiacetal (or hemiketal) carbon, also called the anomeric carbon or reducing carbon. If the structure is analogous to one with the hydroxyl group on the anomeric carbon in the axial position of glucose, then the sugar is an alpha anomer. If, however, that hydroxyl is equatorial, the sugar is a beta anomer. For example, dodecyl β-D-maltoside and dodecyl α-D-maltoside are two cyclic forms of dodecyl maltoside and are anomers. The two different anomers are two distinct chemical structures, and thus have different physical and chemical properties. In one embodiment of the invention, the alkylsaccharide for use with the present invention is a β anomer. In an exemplary aspect, the alkylsaccharide is a β anomer of dodecyl maltoside, tridecyl maltoside or tetradecyl maltoside.

In one embodiment of the present invention, the alkylsaccharide used is a substantially pure alkylsaccharide. As used herein a “substantially pure” alkylsaccharide refers to one anomeric form of the alkylsaccharide (either the α or β anomeric forms) with less than about 2% of the other anomeric form, preferably less than about 1.5% of the other anomeric form, and more preferably less than about 1% of the other anomeric form. In one aspect, a substantially pure alkylsaccharide contains greater than 98% of either the α or β anomer. In another aspect, a substantially pure alkylsaccharide contains greater than 99% of either the α or β anomer. In another aspect, a substantially pure alkylsaccharide contains greater than 99.5% of either the α or β anomer. In another aspect, a substantially pure alkylsaccharide contains greater than 99.9% of either the α or β anomer.

Some exemplary glycosides include maltose, sucrose, and glucose linked by glycosidic linkage to an alkyl chain of 9, 10, 12, 14 or 16 carbon atoms, i.e., nonyl-, decyl-, dodecyl-, tetradecyl- and hexadecyl sucroside, glucoside, and maltoside. As discussed above, these compositions are nontoxic, since they are degraded to long chain alcohols or corresponding long chain fatty acids which are common and normal dietary constituents, and an oligosaccharide. Particular examples include, but are not limited to sucrose cocoate, n-Dodecyl-4-O-α-D-glucopyranosyl-β-D-glucopyranoside (dodecyl-β-D-maltoside) or n-tetradecyl-4-O-α-D-glucopyranosyl-β-D-glucopyranoside (tetradecyl-β-D-maltoside), sucrose laurate, sucrose myristate, sucrose palmitate and mixtures thereof. It is also beneficial if the alkylglycoside chosen is metabolized or eliminated by the body and if this metabolism or elimination is done in a manner that will not be harmfully toxic. Additional saccharides useful in the present invention owing to their safety upon being metabolized in the body include glucose, maltotriose, maltotetraose, and trehalose.

Examples of orally compatible absorption enhancers include alkylsaccharides dodecyl maltoside, n-dodecyl-beta-D-maltoside, tetradecyl maltoside, n-tetradecyl-beta-D-maltoside, tridecyl maltoside, tridecyl-beta-D-maltoside, decyl maltoside, undecyl maltoside, sucrose dodecanoate or sucrose mono-dodecanoate, sucrose tridecanoate or sucrose mono-tridecanoate, sucrose tetradecanoate or sucrose mono-tetradecanoate, sucrose laurate, sucrose myristate, sucrose palmitate and sucrose cocoate which is a mixture of sucrose esters of varying chain lengths from 6 carbons to 18 carbons, with the predominant species in the mixture being sucrose dodecanoate and sucrose tetradecanoate.

The alkylsaccharide of the composition of the invention may be present at a level of from about 0.01% to 20% by weight. More preferred levels of incorporation are from about 0.01% to 5% by weight, from about 0.01% to 2% by weight, or from about 0.01% to 1%. The alkylsaccharide is preferably formulated to be compatible with other components present in the composition.

The compositions described herein are formulated for oral administration. As such, compositions may be formulated in a variety of forms including for example disintegrating capsules, tablets, pills and wafers. Other examples include liquids, syrups, and sprays.

As such, the compositions of the invention can additionally include a pharmaceutically acceptable carrier. A “pharmaceutically acceptable carrier” is an aqueous or non-aqueous agent, for example alcoholic or oleaginous, or a mixture thereof, and can contain a surfactant, emollient, lubricant, stabilizer, dye, perfume, preservative, acid or base for adjustment of pH, a solvent, emulsifier, gelling agent, moisturizer, stabilizer, wetting agent, time release agent, humectant, or other component commonly included in a particular form of pharmaceutical composition. Pharmaceutically acceptable carriers are well known in the art and include, for example, aqueous solutions such as water or physiologically buffered saline or other solvents or vehicles such as glycols, glycerol, and oils such as olive oil or injectable organic esters. A pharmaceutically acceptable carrier can contain physiologically acceptable compounds that act, for example, to stabilize or to increase the absorption of the specific inhibitor, for example, carbohydrates, such as glucose, sucrose or dextrans, antioxidants, such as ascorbic acid or glutathione, chelating agents, low molecular weight proteins or other stabilizers or excipients. A pharmaceutically acceptable carrier can also be selected from substances such as distilled water, benzyl alcohol, lactose, starches, talc, magnesium stearate, polyvinylpyrrolidone, alginic acid, colloidal silica, titanium dioxide, and flavoring agents.

In preparing the compositions for oral dosage form, any of the usual pharmaceutical carriers may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets.

Tablets and capsules represent oral dosage unit forms in some embodiments. If desired, tablets may be coated by standard aqueous or nonaqueous techniques. The amount of active peptide in such therapeutically useful compositions is such that an effective dosage will be obtained. In another advantageous dosage unit form, sublingual constructs may be employed, such as sheets, wafers, tablets or the like.

The tablets, pills, capsules, and the like may also contain a binder such as gum tragacanth, acacia, corn starch or gelatin; excipients such as dicalcium phosphate; a disintegrating agent such as corn starch, potato starch or alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be utilized as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Examples of preservatives that may be used in the compositions of the present invention, include, but are not limited to preservatives such as ethylene diamine tetraacetic acid (EDTA), sodium azide, p-hydroxybenzoate and its analogs, octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, chlorobutanol, m-cresol and alkyglycosides such as dodecyl maltoside.

Proteolysis can be reduced by addition of protease inhibitors such as aprotinin, soybean trypsin inhibitor, and the like. Examples of protease inhibitors include bestatin, amastatin, boroleucin, borovaline, aprotinin, pepstatin A, leupeptin hemisulfate EDTA, EGTA, aminocaproic acid, chymostatin, and alpha-1-antitrypsin, among others. Stabilization in the gastrointestinal tract can also be accomplished by addition of a pH modifier to the drug formulation. Such pH modifiers may raise or lower the pH of the drug formulation. Yet another way to increase stabilization of a peptide in the gastrointestinal tract involves enteric coating, encapsulation, or time release coatings that prevent exposure of the drug formulation to parts of the gastrointestinal tract which may provide a hostile environment or to ensure release in portions of the gastrointestinal tract where peptides may be more stable.

In general, the actual quantity of cyclic peptide administered to a patient will vary between fairly wide ranges depending upon the formulation used, and the response desired. The dosage for treatment is administration, by any of the foregoing means or any other means known in the art, of an amount sufficient to bring about the desired therapeutic effect. In general, cyclic peptides are highly active. For example, a cyclic peptide can be administered at about 0.01, 0.05, 0.1, 0.5, 1, 5, 10, 50, 100, or 500 μg/kg body weight, depending on the specific peptide selected, the desired therapeutic response, the formulation and other factors known to those of skill in the art.

The term “subject” or “patient” as used herein refers to any individual or patient to which a composition is administered. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, and the like, and primates (including monkeys, chimpanzees, orangutans and gorillas) are included within the definition of subject.

In various embodiments, the oral bioavailability of a cyclic peptide is increased by at least 5%, 10%, 15%, 20%, 30%, 40%, 50%, 60%, 70%, 80%, 90%, 100%, 150%, 200%, 250%, 500%, 1000% or greater when administered in the presence of an alkylsaccharide as compared to the same cyclic peptide administered in the absence of the alkylsaccharide.

The present invention is more particularly described in the following examples which are intended as illustrative only since numerous modifications and variations therein will be apparent to those skilled in the art. The following examples are intended to illustrate but not limit the invention.

### Example 1

### Oral Administration of Octreotide

Octreotide is a cyclized and 8-mer peptide with the following sequence that is both cyclized and contains non-natural amino acids. The amino acid sequence is cyclo-D-Phe-Cys-Phe-D-Trp-Lys-Thr-Cys-Thr(ol) (SEQ ID NO: 1) (disulfide bridge cys2-cys7).

Octreotide is an effective option for the medical treatment of patients with acromegaly. It is a synthetic analogue of somatostatin, with similar effects but a prolonged duration of action. Octreotide (acetate) is routinely given by subcutaneous (s.c.) or intramuscular injection. Herein the feasibility of oral delivery of octreotide acetate reconstituted with increasing concentrations (0.5%, 1.5% and 3.0%) of certain alkylsaccharides known to be effective in increasing intranasal transmucosal absorption is described. While these enhancing agents work to increase nasal absorption they have not been found to be equally effective in increasing oral absorption of most peptides. Surprisingly, a small subset or subclass of peptides has been found to undergo oral absorption enhancement using certain alkylsaccharides. The subclass of peptides found to be orally absorbed in the presence of certain alkyl saccharides are generally small peptides having a cyclic structure rather than linear structure and ideally containing non-natural amino acids.

In this example, the pharmacokinetics of orally delivered (by gavage) octreotide acetate in the presence of dodecyl-beta-D-maltoside (DDM) is compared to that of octreotide acetate administered subcutaneously in sodium acetate buffer to male Swiss Webster mice. Oral delivery of octreotide acetate in 0.5% DDM significantly enhances total uptake (1,254.08 ng/ml/min vs. 311.63 ng/ml/min, respectively), serum half-life (1.3 min vs. 52.1 min, respectively), and relative bioavailability (4.0 vs. 1.0, respectively) when compared to delivery by s.c. injection. Higher concentrations of DDM do not further enhance uptake, serum half-life, or bioavailability. These results indicate that oral delivery of octreotide acetate in compositions containing DDM is feasible, and is an effective method of administration which significantly improves uptake, bioavailability and half-life when compared to s.c. injection. Thus, oral delivery of octreotide acetate in compositions containing DDM may have significant potential as a novel, non-invasive approach to the treatment of acromegaly and octreotide-mediated symptoms of carcinoid and VIP-secreting tumors.

Six week-old male Swiss Webster mice weighing approximately 30 g are obtained from Taconic Farms (Germantown, N.Y., USA). The animals are housed three per cage in polycarbonate cages fitted with stainless steel wire lids and air filters, and supported on ventilated racks (Thoren Caging Systems, Hazelton, Pa., USA) in the Albany Medical College Animal Resources Facility. The mice are maintained at a constant temperature (24° C.) with lights on from 07:00 to 19:00 h, and allowed food and water ad libitum until used for uptake studies. Lyophilized octreotide acetate is obtained from BCN (Spain and Polypeptide Laboratories, Torrance, Calif.) and DDM is supplied by Aegis Therapeutics (San Diego, Calif.). For subcutaneous (s.c.) delivery, octreotide acetate is dissolved at a concentration of 30 ug/100 μl in 10 mM sodium acetate buffer containing 0.1% EDTA (pH 4.5). For oral delivery, octreotide acetate is dissolved at a concentration of 30 ug/200 ul in 10 mM sodium acetate buffer containing 0.1% EDTA (pH 4.5) and 0.5%, 1.5% or 3.0% DDM and administered by gavage. At time zero (0), octreotide acetate is delivered subcutaneously or by gavage to each mouse. Following treatment, the mice are transferred to separate cages for the designated time period. Five, 10, 15, 30, 60, 120 or 180 min after octreotide acetate delivery, the mice (n=three per time point) are anesthetized with isoflurane (5%) and exsanguinated by cardiac puncture. Euthanasia is confirmed by cervical dislocation. The blood is collected in sterile nonheparinized plastic centrifuge tubes and allowed to stand at room temperature for 1 h. The clotted blood is rimmed from the walls of the tubes with sterile wooden applicator sticks. Individual serum samples are prepared by centrifugation for 30 min at 2600×g in an Eppendorf™ 5702R, A-4-38 rotor (Eppendorf North America, Westbury, N.Y., USA), The serum samples in each experimental group are pooled and stored frozen until assayed for octreotide acetate content by EIA. Octreotide acetate concentrations in the pooled serum samples are assayed in triplicate with a rat/mouse octreotide enzyme immunoassay assay (EIA) kit obtained from Peninsula Laboratories, LLC (San Carlos, Calif.) according to the instructions supplied by the manufacturer. Serum concentrations of octreotide acetate vs. time following s.c. and oral delivery are plotted using the graphics program SigmaPlot™ 8.0 (SPSS Science, Chicago, Ill., USA). The area under each curve (AUC) is calculated with a function of this program. The lowest AUC value obtained is arbitrarily set at 1.0. Relative bioavailability is determined by comparing all other AUC values to 1.0. The period of time required for the serum concentration of octreotide acetate to be reduced to exactly one-half of the maximum concentration achieved following s.c. or oral administration is calculated using the following formula:

t_{1/2}=0.693/kₑₗᵢₘ where kₑₗᵢₘ represents the elimination constant, determined by plotting the natural log of each of the concentration points in the beta phase of the uptake profiles against time.

Linear regression analysis of these plots resulted in straight lines, the slope of which correlates to the kₑₗᵢₘ for each delivery method. Clearance of octreotide acetate from the plasma following s.c. or oral delivery is calculated from the AUC using the following equation:

CL=Dose/AUC

The apparent volume of distribution of octreotide acetate following s.c. or oral delivery is calculated from its half-life and clearance using the following equation:

t_{1/2}=(0:693×Vd)/CL

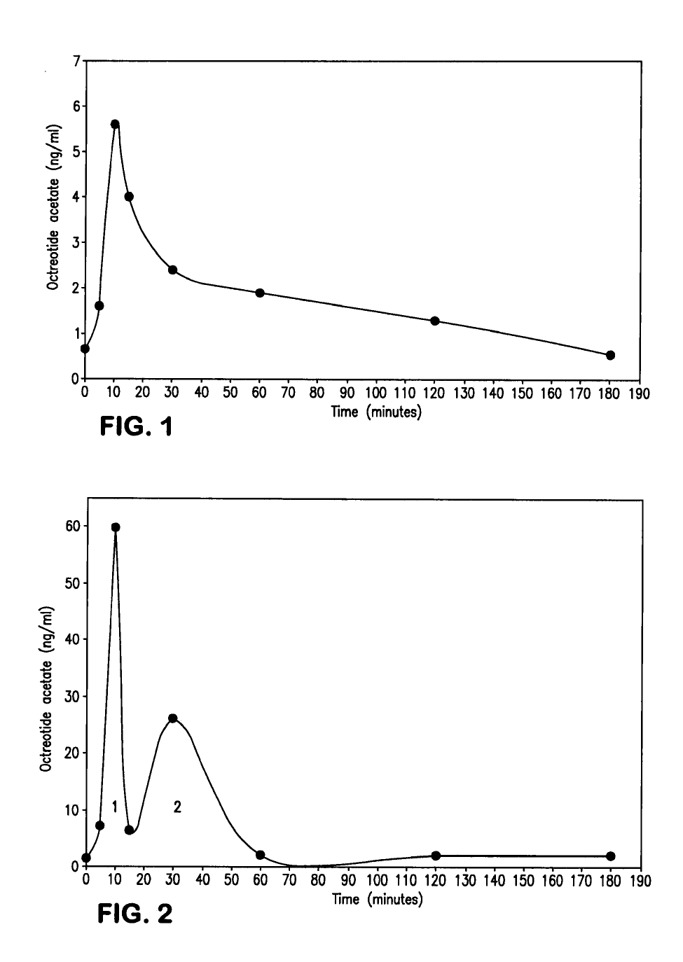

Octreotide acetate uptake profiles following s.c. and oral delivery in 0.5%, 1.5% or 3.0% DDM are shown in FIGS. 1 to 4, respectively. Uptake of octreotide acetate following s.c. delivery showed a single peak at 10 min (tₘₐₓ) with a Cₘₐₓ of 5.6 ng/ml (FIG. 1). Uptake profiles following oral delivery of octreotide acetate in increasing concentrations of DDM are biphasic in nature with an initial peak (Cₘₐₓ₁) at 10 min (tₘₐₓ₁) followed by a second peak (Cₘₐₓ₂) at 30 min (tmax2) (FIGS. 2-4).

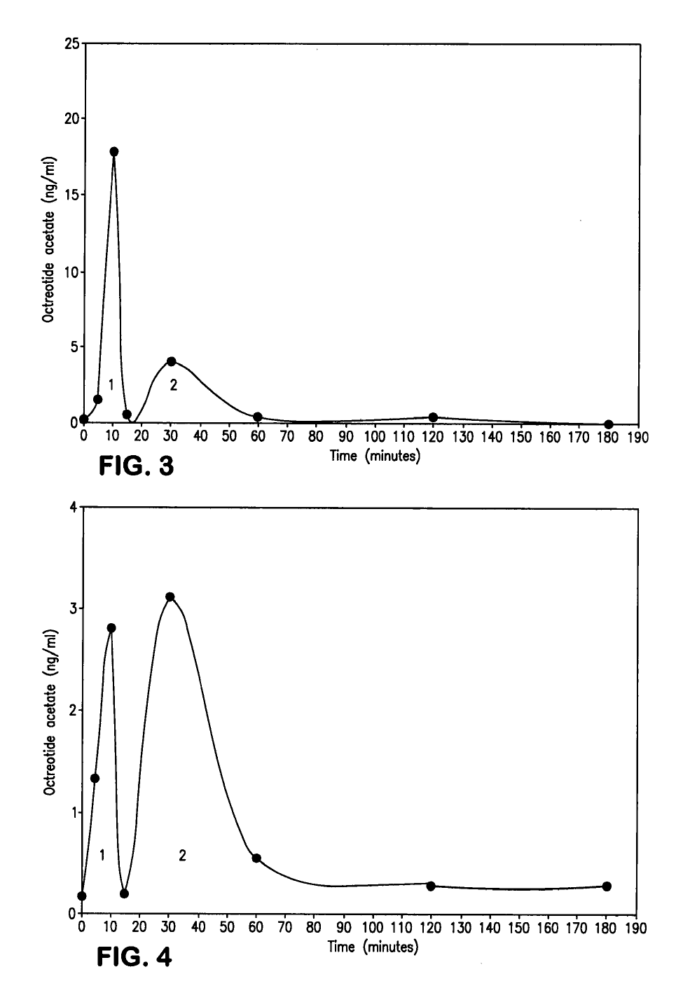

Oral delivery of octreotide acetate in 0.5% DDM produces an uptake profile with a Cₘₐₓ₁ more than 2-fold higher than Cₘₐₓ₂ (59.7 ng/ml vs. 25.9 ng/ml, respectively (FIG. 2). When the DDM concentration is increased to 1.5% and 3.0%, Cₘₐₓ₁ is reduced to 17.8 ng/ml and 3.75 ng/ml, respectively, and Cₘₐₓ₂ is reduced to 4.0 ng/ml and 2.48 ng/ml, respectively (FIGS. 3 and 4). Octreotide acetate concentrations following oral delivery decreased at different rates after each of the two peaks.

The relative bioavailability of octreotide acetate is determined by measuring the area under the uptake curve (AUC) for each delivery method. This value represents the total extent of octreotide acetate absorption into the systemic circulation, or total uptake, following its administration. Because of the biphasic nature of the uptake profiles, the relative bioavailability of octreotide acetate following oral delivery in DDM is determined by measuring the AUC for each of the two peaks in the profile separately, and determined as follows: AUC=AUC₁+AUC₂. The AUC of octreotide acetate after s.c. administration is determined to be 311.63 ng/ml/min, and assigned a relative bioavailability of 1.0. The AUC of octreotide following oral delivery in 0.5%, 1.5% or 3.0% DDM is 1,254.08 ng/ml/min, 230.7 ng/ml/min and 141.24 ng/ml, respectively, and assigned a relative bioavailabilities of 4.0, 0.7 and 0.5.

To determine the serum half-life of octreotide acetate following oral delivery, the kₑₗᵢₘ for each peak in the uptake curves is calculated separately (kₑₗᵢₘ₁ and kₑₗᵢₘ₂). These values are then used to determine the half-life of octreotide acetate under each peak (t_{1/2 1} and t_{1/2 2}). The overall half-life is calculated as follows: t½=t_{1/2 1}+t_{1/2 2}, and determined to be 53.1 min, 25.8 min and 23.6 min following oral delivery in 0.5%, 1.5% or 3.0% DDM, respectively. The serum half-life of octreotide acetate following s.c. delivery is 1.3 min. The present study demonstrates the feasibility of oral administration of octreotide acetate using formulations of octreotide acetate and DDM, a family of patented alkyl saccharide transmucosal absorption enhancing agents that are considered GRAS (Generally Recognized As Safe) substances for oral administration.

### Example 2

### Oral Administration of Lantreotide

Lanreotide is a cyclized and 8-mer peptide with the following sequence that is both cyclized and contains non-natural amino acids. The amino acid sequence is cyclo-H-D-2-Nal-Cys-Tyr-D-Trp-Lys-Val-Cys-Thr-NH2 (SEQ ID NO: 2) (cyclized via disulfide bridge cys2-cys7) where Nal- denotes napthylalanine. The systematic chemical name is: (4S,7S,10S,13R,16S,19S)-10-(4-aminobutyl)-19-[[(2R)-2-amino-3-naphthalen-2-yl-propanoyl]amino]-N-[(1S,2R)-1-carbamoyl-2-hydroxy-propyl]-16-[(4-hydroxyphenyl)methyl]-13-(1H-indol-3-ylmethyl)-6,9,12,15,18-pentaoxo-7-propan-2-yl-1,2-dithia-5,8,11,14,17-pentazacycloicosane-4-carboxamide. This cyclic somatostatin agonist shows a high binding affinity for the somatostatin receptor (SSTR) subtype 2. Lanreotide is obtained from Bachem, Torrance, Calif., and DDM is supplied by Aegis Therapeutics, San Diego, Calif. SDD is supplied by Anatrace, Maumee, Ohio.

The procedure described in Example 1, with the following modifications, is used to determine the relative oral bioavailability compared to s.c. injection of Lanreotide. For subcutaneous (s.c.) delivery, lanreotide acetate is dissolved at a concentration of 90 ug/100 μl in 10 mM sodium acetate buffer containing 0.1% EDTA (pH 4.5), for oral delivery, lanreotide acetate is dissolved at a concentration of 90 ug/200 ul in 10 mM sodium acetate buffer containing 0.1% EDTA (pH 4.5) and 0.5%, 1.5% or 3.0% n-tetradecyl-beta-D-maltoside (TDM) or sucrose dodecanoate (SDD) and administered by gavage, taking the ratios of [oral AUC]/[s.c. AUC].

The relative bioavailabilities of lanreotide in oral formulations containing 0.5%, 1.5% and 3.0% TDM compared to s.c. injection are approximately 4, 1 and <1. The relative bioavailabilities of lanreotide in oral formulations containing 0.5%, 1.5% and 3.0% SDD compared to s.c. injection are approximately 4.5, 2 and <1

### Example 3

### Oral Administration of Cyclized Polypeptides

A class of cyclized peptides containing D-amino acids and non-natural amino acids has been described in U.S. Pat. App Pub. No. 2010/0190710. These peptides generally exhibit poor oral bioavailability. However, in the presence of certain alkyl saccharides, they exhibit significantly enhanced oral bioavailability relative to intravenous (IV) administration.

The compound described is a selective IL-23 receptor antagonist peptide comprising D-amino acids with the formula cyclo-D-TEEEQQYL (SEQ ID NO: 3), where any amino acid is a D-amino acid, MW: ca. 1039, hereafter designated TEEE, supplied by Allostera Pharma as a dry powder, and is formulated for intravenous at 0.02 and 0.2 mg/mL in sterile saline by dilution of an initial stock prepared at 2 mg/mL. Additionally, 4 different formulations of dry TEEE are prepared in gelatin capsules. For each type of dry formulation 10 capsules are prepared to contain enough test article in a way to deliver ca 2.5, 5.0 or 10 mg/kg. The maximum water solubility of TEEE is approximately 10 mg/mL. Solutions and capsules are stored at ca 4° C. upon until dose administration. The intravenous formulation is kept at room temperature pending dose administration. The final appearance of the intravenous formulations consists of clear solutions.

TABLE 1
Test Article Description

| | |
|---|---|
| Test article 1 | TA-1 TEEE (For IV solution administration, |
| | Group 1 and 2) |
| Quantity: | 4.5 mg (initial stock solution prepared at 2.0 mg/mL) |
| Test article 2 | TA-2 TEEE, mannitol in capsule (Group 3-4 and 5) |
| Quantity: | 2.5 mg/kg capsule (10 capsules for Group 3) |
| | 5.0 mg/kg capsule (10 capsules for Group 4) |
| | 10.0 mg/kg capsule (10 capsules for Group 5) |
| Test article 3 | TA-3 TEEE, mannitol, 0.5% (wt/wt) DDM 1 in capsule |
| | (Group 6-7-8) |
| Quantity | 2.5 mg/kg capsule (10 capsules for Group 6) |
| supplied: | 5.0 mg/kg capsule (10 capsules for Group 7) |
| | 10.0 mg/kg capsule (10 capsules for Group 8) |
| Test article 4 | TA-4 TEEE, mannitol, 0.5% (wt/wt) TDM in capsule |
| | (Group 9-10-11) |
| Quantity | 2.5 mg/kg capsule (10 capsules for Group 9) |
| supplied: | 5.0 mg/kg capsule (10 capsules for Group 10) |
| | 10.0 mg/kg capsule (10 capsules for Group 11) |
| Test article 5 | TA-5 TEEE, mannitol, 0.5% (wt/wt) SDD in capsule |
| | (Group 12-13-14) |
| Quantity | 2.5 mg/kg capsule (10 capsules for Group 12) |
| supplied: | 5.0 mg/kg capsule (10 capsules for Group 13) |
| | 10.0 mg/kg capsule (10 capsules for Group 14) |

Male Sprague-Dawley rats are ca 8-9 week old at onset of treatment (weight at onset of treatment, 348-398 g). Animals are allocated into groups of 3 rats/group. Immediately following arrival, animals are uniquely identified via an eartag. Animals are purchased from Charles River Canada (St-Constant, Canada). Following an examination by a qualified In-Life Specialist (including body weight determination), animals are acclimated to the testing facilities for ca 72 hr prior to test article administration. Room conditions are: temperature ca 20° C., humidity ca 50%. Animals are housed in solid bottom polycarbonate cages equipped with a filter top to avoid contamination, fed with standard certified rodent food (brand and lot no. to be documented in the raw data). The animals had continuous access to RO/UV water, via bottles. All rats are administered the compounds intravenously (Groups 1 and 2) in a jugular vein under light isoflurane anesthesia or orally by capsule (one per animal) using a specially designed capsule delivery tube (Groups 3-14). The dose volume is 5.0 ml/kg for groups 1 and 2 and one capsule/animal for Groups 3 to 14. Prior to dosing, each animal is weighed and the weight recorded. Animals are fasted overnight prior to dose administration until 4 hours post-dose administration. Following administration, blood samples (ca 100 uL) are obtained from a jugular vein under light isoflurane anesthesia (except for the last time point collected by cardiac puncture or abdominal vena cava) from each animal at each time point defined in the abbreviated study design below. All blood samples are collected (under light isoflurane anesthesia) into EDTA coated tubes. All blood samples are placed on ice pending centrifugation (at 3200 g for 10 min at ca 4° C.) within 1 hour following collection. Following centrifugation, exactly 15 uL of plasma will be separated by calibrated pipette, transferred into appropriately labeled 96 well plates and stored on dry ice and/or ca −80° C. until assay. Aliquots of 0.5 mL of remaining dosing formulation (after dosing the last animal subject), are transferred into polypropylene tubes and stored on dry ice pending assay. All animals are discarded following collection of the last sample. Plasma concentrations of each compound are determined using an LCMS assay (non-GLP) with an analytical range of ca 25-1000 ng/mL. Non-compartmental pharmacokinetic analysis and bioavailability calculations are performed from individual plasma concentration values at each time point.

TABLE 2
Abbreviated Study Design

| | | | Dose | Dose | Dose | Bleed Schedule | Animal |
|---|---|---|---|---|---|---|---|
| Grp | Compound | Route | (mg/kg) | volume | Conc. | (time in hours) | No. |
| 1 | TEEE | IV | 0.1 | 5 mL/kg | 0.02 mg/ml | 0.08, 0.33, 1, | 1001-1003 |
| 2 | | | 1.0 | | 0.2 ng/mL | 2, 4 6, 8 and 24 | 2001-2003 |
| 3 | TEEE + | PO | 2.5 | 1 capsule/rat | 2.5 mg/kg | 0.25, 0.5, 1, | 3001-3003 |
| | mannitol | | | | capsule | 2, 4, 6, 8 | |
| 4 | | | 5.0 | | 5.0 mg/kg | and 24 | 4001-4003 |
| | | | | | capsule | | |
| 5 | | | 10.0 | | 10.0 mg/kg | | 5001-5003 |
| | | | | | capsule | | |
| 6 | TEEE + | | 2.5 | | 2.5 mg/kg | | 6001-6003 |
| | mannitol + | | | | capsule | | |
| 7 | DDM | | 5.0 | | 5.0 mg/kg | | 7001-7003 |
| | | | | | capsule | | |
| 8 | | | 10.0 | | 10.0 mg/kg | | 8001-8003 |
| | | | | | capsule | | |
| 9 | TEEE + | | 2.5 | | 2.5 mg/kg | | 9001-9003 |
| | mannitol + | | | | capsule | | |
| 10 | TDM | | 5.0 | | 5.0 mg/kg | | 10001-10003 |
| | | | | | capsule | | |
| 11 | | | 10.0 | | 10.0 mg/kg | | 11001-11003 |
| | | | | | capsule | | |
| 12 | TEEE + | | 2.5 | | 2.5 mg/kg | | 12001-12003 |
| | mannitol + | | | | capsule | | |
| 13 | SDD | | 5.0 | | 5.0 mg/kg | | 13001-13003 |
| | | | | | capsule | | |
| 14 | | | 10.0 | | 10.0 mg/kg | | 14001-14003 |
| | | | | | capsule | | |

TABLE 3
Body Weights

| Animal | Body weight (g) |
|---|---|
| 1001 | 366 |
| 1002 | 351 |
| 1003 | 370 |
| 2001 | 370 |
| 2002 | 349 |
| 2003 | 366 |
| 3001 | 348 |
| 3002 | 351 |
| 3003 | 357 |
| 4001 | 357 |
| 4002 | 379 |
| 4003 | 376 |
| 5001 | 379 |
| 5002 | 362 |
| 5003 | 370 |
| 6001 | 354 |
| 6002 | 377 |
| 6003 | 398 |
| 7001 | 375 |
| 7002A | 394 |
| 7003 | 368 |
| 8001 | 366 |
| 8002 | 370 |
| 8003 | 365 |
| 9001 | 362 |
| 9002 | 369 |
| 9003 | 361 |
| 10001 | 348 |
| 10002 | 375 |
| 10003 | 364 |
| 11001 | 379 |
| 11002 | 366 |
| 11003 | 390 |
| 12001 | 382 |
| 12002 | 386 |
| 12003 | 361 |
| 13001 | 355 |
| 13002 | 392 |
| 13003 | 363 |
| 14001 | 368 |
| 14002 | 390 |
| 14003 | 383 |

No clinical signs are observed during the conduct of this study, suggesting relative safety of the TEEE formulations administered orally or intravenously over the range of doses investigated. Relative bioavailbilities compared with iv administration is shown below in Table 4.

TABLE 4
Relative Bioavailabilities of Oral Delivery vs. IV Delivery

| | | | | Dose volume | | Dose | | |
|---|---|---|---|---|---|---|---|---|
| | | | | mL/kg (IV) | | Concentration | | |
| | | | Dose | or capsules/ | | mg/Ml (IV) or mg/kg | Bleed Schedule | Average |
| Group | Compound | Route | (mg/kg) | rat (PO) | Format | capsule (PO) | (time in hours) | Bioavailabillty |
| 1 | TEEE in saline | IV | 0.1 | 5 | Liquid | 0.02 | 0.08, 0.33, 1, 2, | |
| 2 | | IV | 1 | 5 | Liquid | 0.2 | 4 6, 8 and 24 | |
| 3 | TEEE + | PO | 2.5 | 1 | Capsule | 2.5 | 0.25, 0.5, 1, 2, | 42.1% |
| 4 | mannitol | PO | 5 | 1 | Capsule | 5 | 4, 6, 8 and 24 | n.d. |
| 5 | | PO | 10 | 1 | Capsule | 10 | | 32.1% |
| 6 | TEEE + | PO | 2.5 | 1 | Capsule | 2.5 | | ad. |
| 7 | mannitol + DDM | PO | 5 | 1 | Capsule | 5 | | 39.7% |
| 8 | (0.5% wt/wt) | PO | 10 | 1 | Capsule | 10 | | 27.1% |
| 9 | TEEE + | PO | 2.5 | 1 | Capsule | 2.5 | | 61.5% |
| 10 | mannitol+ TDM | PO | 5 | 1 | Capsule | 5 | | n.d. |
| 11 | (0.5% wt/wt | PO | 10 | 1 | Capsule | 10 | | 69.7% |
| 12 | TEEE + | PO | 2.5 | 1 | Capsule | 2.5 | | 78.9% |
| 13 | mannitol + SDD | PO | 5 | 1 | Capsule | 5 | | 72.2% |
| 14 | (0.5% wt/wt) | PO | 10 | 1 | Capsule | 10 | | n.d. |

### Example 4

### Oral Administration of Cyclized Polypeptides

A cyclic peptide with molecular weight of 969 Daltons containing non-natural amino acids reported by Mesfin et al. (J Pept Res, 58 (3) 246-256 (2001)) has been demonstrated to exhibit some oral bioavailability by measuring the pharmacodynamic properties of prevention of tumor growth in human breast cancer xenografts in rats upon oral administration. The peptide has the sequence cyclo-EKTXVNXGN (SEQ ID NO: 4), where X is the unnatural amino acid hydroxyproline (hereafter designated EKTX). Oral bioavailability of EKTX in comparison to s.c. injection is enhanced upon addition of various alkylsaccharides.

In this example, the pharmacokinetics of orally delivered (by gavage) EKTX in the presence of dodecyl-beta-D-maltoside (DDM), tetradecyl-beta-D-maltoside (TDM), and sucrose dodecanoate (SDD) are compared to that of EKTX acetate administered subcutaneously in sodium acetate buffer to male Sprague-Dawley rats (approx. age 80 days), 3 rats per group. Sprague-Dawley rats are acquired from Taconic Farms in Germantown, N.Y. The animals are allowed to adjust to the facility for five days. Rats are maintained at a constant temperature (24° C.) under a 12 h light-dark cycle with lights out at 7 p.m., and fed a standardized diet of RMH3000 and filtered water ad libitum. A group of 10 rats are given 100 μg EKTX in saline s.c. A second, third, and fourth group of ten rats each is given 200 μg of EKTX containing 0.5% DDM, TDM or SDD respectively administered by oral gavage. EKTX is prepared as described by Mesfin et al (2001). DDM and TDM is supplied by Aegis Therapeutics, San Diego, Calif.) and SDD is supplied by Anatrace Inc., Maumee, Ohio. For subcutaneous (s.c.) delivery, EKTX acetate is dissolved at a concentration of 100 ug/100 μl in 5 mM sodium acetate buffer containing 0.1% EDTA (pH 5.5). For oral delivery, EKTX acetate is dissolved at a concentration of 200 ug/200 ul in 5 mM sodium acetate buffer containing 0.1% EDTA (pH 5.5) and 0.5% DDM, TDM or SDD and administered by gavage to each rat lightly anesthetized with isoflurane (5%). At time zero (0), EKTX is delivered subcutaneously or by gavage to each rat.

Following treatment, the rats are transferred to separate cages for the designated time period. Five, 10, 15, 30, 60, 120 or 180 min after EKTX delivery blood is drawn from the tail vein, and plasma is immediately prepared from each blood sample using lithium/heparin as the anticoagulant. Individual plasma samples are prepared by centrifugation for 30 min at 2600×g in an Eppendorf 5702R, A-4-38 rotor (Eppendorf North America, Westbury, N.Y., USA). The serum samples in each experimental group are stored frozen until assayed for EKTX content by ELISA. Dosing solutions are also stored frozen until analyzed by ELISA. Serum levels of EKTX are measured using a competitive ELISA assay as follows. Rabbit antibodies are prepared by coupling EKTX to limpet hemocyanin using glutaraldehyde to create an immunogen and injecting it into rabbits on a weekly basis, the first injection incorporating Complete Freund's Adjuvant, and subsequent injections employing Incomplete Freund's Adjuvant. After ten weeks, serum is collected and the IgG fraction is prepared by chromatographic separation on a Protein A sepharose column. Horseradish peroxidase labeled EKTX is prepared using the sodium periodate cyanoborohydride reduction method described by Tresca et al. (Ann Biol Clin, 53 (4):227-31 (1995)). Rabbit anti-EKTX is coated in a 96 well microtiter plate, and ELISA assay conditions are optimized as described in Maggio (Enzyme-Immunoassay. Boca Raton, Fla.: CRC Press, Inc. 1980). Pharmacokinetic parameters are calculated from the plasma concentration-time data. The ratio of the AUC (area under the curve) in the presence of the designated alkylsaccharides compared to the AUC without alkylsaccharide provides a measure of the enhancement of oral bioavailability. All three alkylsaccharides tested, DDM, TDM and SDD, increase the oral absorption of EKTX by a factor of approximately 1.4 to 1.9.

Although the invention has been described with reference to the above examples, it will be understood that modifications and variations are encompassed within the spirit and scope of the invention. Accordingly, the invention is limited only by the following claims.

What is claimed is:

1. An oral composition comprising:
a) a cyclic peptide; and
b) at least one alkylsaccharide absorption enhancer.

2. The oral composition of claim 1, wherein the alkylsaccharide has an alkyl chain including between 10 to 16 carbons.

3. The oral composition of claim 1, wherein the alkylsaccharide is selected from the group consisting of sucrose cocoate, n-dodecyl-beta-D-maltoside, n-tetradecyl-beta-D-maltoside, sucrose laurate, sucrose myristate, sucrose palmitate, tridecyl-beta-D-maltoside, sucrose mono-dodecanoate, sucrose mono-tridecanoate, and sucrose mono-tetradecanoate.

4. The oral composition of claim 1, wherein the cyclic peptide comprises 50 or less amino acids.

5. The oral composition of claim 1, wherein the cyclic peptide comprises from 2 to 50 amino acids.

6. The oral composition of claim 1, wherein the cyclic peptide comprises from 3 to 20 amino acids.

7. The oral composition of claim 1, wherein the cyclic peptide comprises 5 to 15 amino acids.

8. The oral composition of claim 1, wherein the cyclic peptide comprises at least one non-natural amino acid.

9. The oral composition of claim 8, wherein the at least one non-natural amino acid is a D-amino acid.

10. The oral composition of claim 1, wherein the at least one D-amino acid is D-phenylalanine or D-tryptophan.

11. The oral composition of claim 8, wherein the at least one non-natural amino acid is selected from the group consisting of hydroxyproline, napthylalanine, norleucine, tert-leucine, hydroxyvaline, allothreonine, beta-dialkylserine, cyclohexylalanine, allylglycine, pyridylalanine, 4-hydroxymphenylglycine, phenylglycine, homoserine, 3,4,dihydroxyphenylalanine, and 4-chlorophenylalanine.

12. The oral composition of claim 1, wherein the cyclic peptide is an antibiotic.

13. The oral composition of claim 1, wherein the antibiotic is selected from the group consisting of daptomycin, vancomycin, bacitracin, gramicidin, grandamycin, viomycin, capreomycin, microcin J25, bacteriocin AS-48, rhesus theta defensin-1 (RTD-1), streptogramins and polymyxins.

14. The oral composition of claim 1, wherein the cyclic peptide is selected from SEQ ID NOs: 1-4.

15. The oral composition of claim 1, further comprising a mucosal delivery-enhancing agent selected from the group consisting of an aggregation inhibitory agent, a charge-modifying agent, a pH control agent, a degradative enzyme inhibitory agent, a mucolytic or mucus clearing agent, a chitosan, and a ciliostatic agent.

16. The oral composition of claim 1, further comprising benzalkonium chloride or chloroethanol.

17. The oral composition of claim 1, further comprising an agent selected from the group consisting of a buffering agent, a surfactant, a bile salt, a phospholipid additive, a mixed micelle, a liposome, a carrier, an alcohol, an enamine, a nitric oxide donor compound, a long-chain amphipathic molecule, a small hydrophobic penetration enhancer, a sodium or a salicylic acid derivative, a glycerol ester of acetoacetic acid, a cyclodextrin or beta-cyclodextrin derivative, a medium-chain fatty acid, a chelating agent, an enzyme degradative to a selected membrane component, a modulatory agent of epithelial junction physiology, a vasodilator agent, and a selective transport-enhancing agent.

18. The oral composition of claim 1, further comprising at least one excipient selected from the group consisting of bulking agents, tableting agents, dissolution agents, wetting agents, lubricants, colors, flavors, disintegrants, coatings, binders, antioxidants, taste masking agents and sweeteners.

19. The oral composition of claim 18, wherein the bulking agent is mannitol, sorbitol, sucrose, or trehalose.

20. The oral composition of claim 1, wherein the composition is formulated as a orally disintegrating capsule, tablet, pill or wafer.

21. The oral composition of claim 1, wherein the composition is formulated as a liquid, syrup, or spray.

22. A method of increasing the oral bioavailability of a linear peptide comprising:
a) cyclizing a linear peptide to form a cyclic peptide; and
b) orally administering the cyclized peptide in the presence of at least one alkylsaccharide absorption enhancer to a subject.

23. The method of claim 22, wherein the cyclic peptide and the at least one alkylsaccharide are admixed to form a composition prior to administration.

24. The method of claim 23, wherein the alkylsaccharide has an alkyl chain including between 10 to 16 carbons.

25. The method of claim 24, wherein the alkylsaccharide is selected from the group consisting of sucrose cocoate, n-dodecyl-beta-D-maltoside, n-tetradecyl-beta-D-maltoside, sucrose laurate, sucrose myristate, sucrose palmitate, tridecyl-beta-D-maltoside, sucrose mono-dodecanoate, sucrose mono-tridecanoate, and sucrose mono-tetradecanoate.

26. The method of claim 22, wherein the cyclic peptide comprises 50 or less amino acids.

27. The method of claim 26, wherein the cyclic peptide comprises from 2 to 50 amino acids.

28. The method of claim 26, wherein the cyclic peptide comprises from 3 to 20 amino acids.

29. The method of claim 26, wherein the cyclic peptide comprises 5 to 15 amino acids.

30. The method of claim 22, wherein the cyclic peptide comprises at least one non-natural amino acid.

31. The method of claim 30, wherein the at least one non-natural amino acid is a D-amino acid.

32. The method of claim 31, wherein the at least one D-amino acid is D-phenylalanine or D-tryptophan.

33. The method of claim 30, wherein the at least one non-natural amino acid is selected from the group consisting of hydroxyproline, napthylalanine, norleucine, tert-leucine, hydroxyvaline, allothreonine, beta-dialkylserine, cyclohexylalanine, allylglycine, pyridylalanine, 4-hydroxymphenylglycine, phenylglycine, homoserine, 3,4,dihydroxyphenylalanine, and 4-chlorophenylalanine.

34. The method of claim 22, wherein the cyclic peptide is an antibiotic.

35. The method of claim 34, wherein the antibiotic is selected from the group consisting of daptomycin, vancomycin, bacitracin, gramicidin, grandamycin, viomycin, capreomycin, microcin J25, bacteriocin AS-48, rhesus theta defensin-1 (RTD-1), streptogramins and polymyxins.

36. The method of claim 22, wherein the cyclic peptide is selected from SEQ ID NOs: 1-4.

37. The method of claim 23, wherein the composition further comprises a mucosal delivery-enhancing agent selected from the group consisting of an aggregation inhibitory agent, a charge-modifying agent, a pH control agent, a degradative enzyme inhibitory agent, a mucolytic or mucus clearing agent, a chitosan, and a ciliostatic agent.

38. The method of claim 23, wherein the composition further comprises benzalkonium chloride or chloroethanol.

39. The method of claim 23, wherein the composition further comprises an agent selected from the group consisting of a buffering agent, a surfactant, a bile salt, a phospholipid additive, a mixed micelle, a liposome, a carrier, an alcohol, an enamine, a nitric oxide donor compound, a long-chain amphipathic molecule, a small hydrophobic penetration enhancer, a sodium or a salicylic acid derivative, a glycerol ester of acetoacetic acid, a cyclodextrin or beta-cyclodextrin derivative, a medium-chain fatty acid, a chelating agent, an enzyme degradative to a selected membrane component, a modulatory agent of epithelial junction physiology, a vasodilator agent, and a selective transport-enhancing agent.

40. The method of claim 23, wherein the composition further comprises at least one excipient selected from the group consisting of bulking agents, tableting agents, dissolution agents, wetting agents, lubricants, colors, flavors, disintegrants, coatings, binders, antioxidants, taste masking agents and sweeteners.

41. The method of claim 40, wherein the bulking agent is mannitol, sorbitol, sucrose, or trehalose.

42. The method of claim 23, wherein the composition is administered as an orally disintegrating capsule, tablet, pill or wafer.

43. The method of claim 23, wherein the composition is administered as a liquid, syrup, or spray.
